(19) United States
(12) Patent Application Publication
(10) Pub. No.: US 2010/0047352 A1
(43) Pub. Date: Feb. 25, 2010

(54) COMPOSITIONS AND METHODS FOR TREATING EXCESSIVE BLEEDING

(75) Inventors: Allan D. Pronovost, San Diego, CA (US); Henry Eisenson, San Diego, CA (US)

Correspondence Address: FOLEY & LARDNER LLP, 111 HUNTINGTON AVENUE, 26TH FLOOR, BOSTON, MA, 02199-7610, US

(73) Assignee: Hemo Nanoscience, LLC

(21) Appl. No.: 12/412,171

(22) Filed: Mar. 26, 2009

Related U.S. Application Data
(63) Continuation of application No. 11/187,337, filed on Jul. 22, 2005, now abandoned.
(60) Provisional application No. 60/590,214, filed on Jul. 22, 2004.
(60) Provisional application No. 60/590,845, filed on Jul. 23, 2004.

(51) Int. Cl. A61K 9/14; A61K 33/00; A61K 38/43; A61K 38/48; A61P 7/04

(52) U.S. Cl. 424/489; 424/724; 424/94.1; 424/94.64

(57) ABSTRACT

The inventive material is a unique family of externally used wound sealants based upon a binding agent of reactive submicron silica particles that, when hydrated, agglomerate in the form of a supramolecular cross-linked network serving as the structural framework facilitating clot formation. A thrombolytic cascade accelerant can be provided, optionally with additional clotting factors, to further accelerate the clotting process.

### RELATED APPLICATIONS

This application claims priority to U.S. Ser. No. 60/590,214 filed Jul. 22, 2004 and U.S. Ser. No. 60/590,845 filed Jul. 23, 2004, the entirety of these applications are hereby incorporated herein by reference.

### FIELD OF THE INVENTION

The present invention relates generally to wound sealants with several applications: simple external vascular bleeding; external deep wound trauma sites to reduce, control, or eliminate bleeding or control additional bleeding; and internal bleeding applications.

### BACKGROUND

Wound sealants have been in use for years, in many forms, with varying degrees of suitability to various classes of wounds. Typically, wound sealants for hemostatic control are typically 2 or 3 step multi-component formulations mixed prior to use and allowed to set before application. Wound sealants formulations of materials purified from human or animal blood or tissue products are typically slow to react (>30 min) and generally ineffective against pressure bleeding or recurrent bleeding.

Certain types of wound sealants known in the art rely upon the use of fibrinogen and thromboplastin (WO 97297972), fibrinogen and thrombin (U.S. Pat. No. 5,219,328), fibrin analogs (U.S. Pat. No. 5,292,333), fibrinogen analogs and thrombin (U.S. Pat. Nos. 5,645,849 and 5,643,596), thrombin alone (0277016B1), thrombin and thromboplastin and Factors VII, IX, and X with biosealant adhesive/glue (gelatin/resorcinol/glutaraldehyde) (U.S. Pat. No. 6,168,788), and even adhesives alone such as methylacrylate (U.S. Pat. No. 5,981,621, U.S. Pat. No. 6,607,631, and others). The FDA disapproved the use of human fibrinogen in 1978 putting an end to the use of animal or human plasma derived material as required in U.S. Pat. No. 6,168,788. In addition the purification methods described in U.S. Pat. No. 6,168,788 are inadequate in that they result in the co-purification of a variety of other blood factors that should not be present in the mixture (other thrombolytic cascade factors) that may likely initiate the cascade slowly over time, in addition to copurification of other materials not desired such as albumins, immunoglobulin, viruses, and the like. Animal derived materials are also problematic in that they can foster histocompatibility problems in the host recipient in addition to containing viruses and prions. In the above cited patents, the fibrinogen component typically supplied the “glue” and the thrombin or thromboplastin component supplied the “activator” for the clotting process. Those formulations employing thromboplastin only as activator rely on the application site to provide prothrombin to initiate the clotting cascade. This enzymatic material if present occurs in low concentrations naturally and may be readily exhausted. Those formulations that employ thrombin may initiate immediate clotting with fibrinogen supplied by the application site but lack the ability to convert additional prothrombin to thrombin in the event of additional bleeding.

Other multi-step bioactive wound sealants have included: addition of plasmin inhibitors (U.S. Pat. No. 5,645,859); addition of polyol stabilizers (EP0277096B1); addition of oxyacid salts coupled with insoluble cation exchange material and hydrophilic polymers (20020141964); calcium control (U.S. Pat. No. 5,318,524); pH control (U.S. Pat. No. 5,219,328); synthetic prothrombin converter usage (PAJ10052267); or thrombin-like proteases (EPS708067A1).

In addition, several fiber-based products such as sterilized spider webs have been used as matrices to aid clotting, in addition to plastic webbing applied to wound sites as a dressing, use of clot accelerants such as aminocaproic acid, and use of various astringents such as alum have been used to constrict capillaries at the site and aid clotting.

Two-step cyanoacrylates (Closure Medical) or hydrophilic polymers like carboxymethylcellulose or Chitosan (2002141964) are oftentimes applied to the surface of the fluid at the wound site to form a film above the clot located underneath (as a covering generally not interactive with the wound).

U.S. Pat. No. 6,060,461 describes a topical powder clotting material not interactive with the wound site that employs porous particles (epiclorhydrin cross-linked agarose i.e., Sephadex (Pharmacia) which is a research material not suitable for human use and considered toxic according to MSDS); the Sephadex used comprises 50 nm particles that are hydrophilic yet porous (not solid) that swell in the presence of liquid due to gel rehydration (like spaghetti). U.S. Pat. No. 6,386,203 describes a dermal adhesive using fumed silica of 10-100 nm size; the material used however is hydrophobic containing methyl groups and is not hydrophilic. It is used in conjunction with cyanoacrylate to literally prevent the dermal adhesive from dropping into wound site crevices so as to preserve the feature of an above-the-wound dressing based on it's hydrophobic properties. U.S. Pat. No. 4,373,519 describes a wound dressing primarily employing absorbent particles (porous clay, chitosan). Conventional, chemically-inert, solid, macroparticulate, non-absorbent, sieved silica is added as an inert filler with no functionality ascribed to it other than filler. U.S. Pat. No. 5,741,509 describes a water impermeable, non-wound-fluid-interactive, topical grease dressing similar in function to U.S. Pat. No. 6,386,203, composed of a solvent-based grease of silicone oil and hydrophobic fumed silica wherein the solvent in the formulation evaporates and leaves a waterproof layer on top of the wound. The fumed silica used is completely hydrophobic and does not interact with the fluid at the wound site again serving the purpose of a filler. US 20020128336 refers to a non-medical (non-wound) adhesive as used in the building industry (caulk); described is a waterproof silica caulk adhesive (foam-like) composed of solid macroparticulate silica, titanium, and alumina for use in bathrooms. It employs 10-50 nm hydrophobic, non-interactive and non-absorbent silica. US 20030133990 describes the use of porous molecular sieves of a naturally occurring, calcium enriched, clay (Zeolite) that act as absorbent to dry a wound site. To aid this drying further, conventional beaded (1 mm), porous, silica gel desiccant material (as routinely found as desiccant in consumer goods for moisture control) is added. This additional material comprises macrobeads which are porous which makes it hydroscopic as an absorbent. It is comprised of chemically inert, beaded, silica gel as desiccant. The porosity affords the absorbent function for this hydroscopic material. There is no reactive surface chemistry.

As such the problem with conventional wound sealants that are synthetic biosealants is that they tend to harden the initial clot like plaster of Paris; furthermore, once hardened the sealants are generally spent (can no longer react to bleeding). Conventional wound sealants are not generally effective against pressure bleeding, heavy trauma, deep wound sites, prolonged bleeding, rebleeding, bleeding due to hemophilia, bleeding by a patient using blood thinning agents, or clot disruption resulting from simple body movement.

Other wound sealant methods include multiple components that must be delivered to the wound site separately (multiple-delivery) due to potential interaction in storage. These are not one step formulations. While these techniques may be reasonably suitable for the particular purposes they were developed to address, they are generally compromises. There remains a need in the art for improved wound sealant compositions.

### SUMMARY OF THE INVENTION

The main deficiency of conventional wound sealants is a failure to present an optimized combination of speed of clotting, effectiveness under pressure bleeding conditions, and clots that are dynamic over time in response to the needs of the trauma site. For example, typical wound sealants do not also function as a penetrative, interactive pliable and remaleable wound dressing but, rather, are used in conjunction with separate wound dressings or as noted above applied to the fluid surface above and away from the clot itself as an attempt to glaze or seal over the wound.

The ideal wound sealant would afford the following properties: “true”one-step formulation and delivery; no pre-wetting, mixing or activation time; augment and accelerate natural clotting processes; utilize materials provided by the body at the wound site in response to amount and type of bleeding (intermittent, recurrent, pressure); use of critical “activators” from the two main reactions of the Extrinsic Cascade for clotting; control immediate and sustained bleeding; provide lattice web formation in situ after application based on it's reactive (interactive) properties; serve as a dynamic pliable and malleable wound dressing; composed of bioactive materials of non animal origin free of viruses; use of thrombolytic activators not reactive with each other during storage, over time, or after application to allow separate functions for immediate and sustained bleeding control; supply of clot activating factors “in excess” when body itself may limit supply or supply may be spent on initial bleeding; and are of a stable formulation with long shelf life.

In these respects, the wound sealant according to the present invention, uses clot accelerant lattice technology, comprised of reactive silica nanoparticles, and optional recombinant thrombolytic factors, substantially departs from the conventional concepts and designs of the prior art, and in so doing provides a material that improves the performance of wound sealants and clotting enhancers. In the present invention, the use of genetically engineered thrombin and thromboplastin by recombinant cloning and expression of the active peptides yields stable bioactive preps free of other thrombolytic factors and other blood products, dangerous blood borne viruses like HIV, immunoglobulins, cytokines and the like.

The basic clot accelerant is composed of short chains of non-porous silica nanoparticles which contain a very high density of highly reactive, hydrophilic surface hydroxyl groups which upon contact with fluid at the wound site, instantly cross-link by hydrogen bonding in water. Reactive silica nanoparticles employ ‘nano’ technology based on their extremely small size of particles (as small as viruses) and their extremely high surface area.

This is not conventional ‘micro’ technology silica which is chemically inert having no functional hydroxyl groups. Conventional silica is approximately 1,000-fold larger in size (macro- or micron-based beads). It is also non-hydrophilic and non-lattice forming. Reactive nanoparticles, on the other hand, readily hydrogen bond with each other directly or through polar water molecules as an intermediary to create a three dimensional structural labyrinth at the wound site and cause thixotropy (thickening) of the ambient aqueous fluid (serum) to create a lattice in situ. Viscosity increase and thixotropy development are both the direct result of three dimensional labrinyth formation as a result of hyrdrogen bonding. The lattice reforms continually upon shearing in response to movement and shear forces which cause dynamic reassociation, thus resulting in a flexible wound sealant matrix of hydroxysilica nanoparticles.

This fibrin-independent lattice formed in situ serves as the backbone for natural clot formation. In addition, the formulation may contain plasma-derived (only useful for veterinary applications), or preferably recombinant human thrombin and thromboplastin, key activators of the two major reactions of the Extrinsic Cascade. Thrombin acts with fibrinogen to form the final clot and facilitates “immediate” clot formation, whereas Thromboplastin acts with prothrombin to initiate the above reaction or reinitiate it for clotting upon sustained or recurrent bleeding. Collectively, the combination of these materials present substantial advantages over, and avoid deficiencies in, known methods and substances. The general purpose of the present invention, which will be described subsequently in greater detail, is to provide a new wound sealant composition that forms a clot-accelerating lattice, that in certain embodiments includes one or more optional recombinant thrombolytic activators that independently modulate the clotting pathway to staunch immediate and sustained bleeding. The composition provides many advantages over the existing wound sealants mentioned herein.

The present invention is generally discussed in nonlimiting aspects which give rise to numerous embodiments and product formulations specific to various first-aid and more serious medical applications. In one aspect, the present invention provides a composition of hydroxylated silica nanoparticles, that when applied to a wound site will polymerize to form a hydrogen bonded clot-accelerant lattice. The hydroxylated silica nanoparticles are also referred herein as binding agents, and the preparation can be applied directly to the wound site to staunch simple vascular bleeding (cuts and scrapes). Silica (silicon dioxide) particles that are small enough to have surface area as high as 500 M²/g or more (hereinafter “nanoparticles”) are preferred as the binding agents. The surface area range may be from 25 to 500 M²/g, preferably between 175 to 300 M²/g. Such nanoparticles are extremely small (from about 0.01 nanometers to about 1 micrometer in diameter, more preferably 0.1 to 100 nanometers in diameter, and most preferably 1 to 50 nanometers in diameter) with a maximum of 0.02% 325 mesh residue (44 microns) present in the preparation. The small size coupled with the large surface area allows for an excessive number of reactive hydroxyl groups to facilitate cross linking in the highly polar water environment.

Silica nanoparticles that are suitable for the present invention are typically formed by the common industrial “fumed silica” process which involves heating to over 1800° C. These silica nanoparticles are hydroxylated as a direct result of the fuming process, and the appropriate hydroxysilica nanoparticles that can be used as binding agents are not to be confused with larger chemically-inert silica macro- or microparticles (greater than 1 micrometer in diameter), which are produced by grinding and sieving, and are commonly used in the food industry for anti-caking purposes. The conventional larger silica particles lack the necessary active hydroxyl functional groups on the surface of the particle.

These binding agents promote rapid clot formation upon contact with fluid at the wound site when the highly hydrophilic silica nanoparticles containing hydroxyl surface groups instantly cross-link to form a hydrogen bonded lattice. The binding agents hydrogen bond with each other or to polar water molecules as an intermediary in the lattice. The water molecules participate in the bonding reaction between adjacent hydroxysilica nanoparticles. Upon primary hydration at the wound site the binding agent assumes a thixotrophic state over time. The degree of thixotropy and thickening of the fluid is directly proportional to the density of nanoparticles in the fluid and both the concentration and the formulation composition (pH, additives) can be adjusted to optimize the viscosity, thickening, flow, and movement of the sample. The binding agent, now assembled as a hydrogen bonded lattice, becomes integrated throughout the wound, forming a barrier to blood loss but not impeding the function of the subject's intrinsic clotting factors supplied and activated by the bleeding itself.

In another aspect, the binding agents include at least one and preferably two enzymes or active fragments thereof, for example clotting agents including thrombolytic activators of the Extrinsic Pathway. Thrombolytic cascade accelerants suitable for use herein include the key extrinsic pathway activators human thrombin and thromboplastin. Thrombin combines with fibrinogen to form the clot and facilitates “immediate” clot formation at the wound site, whereas thromboplastin combines with prothrombin to initiate the second reaction above or reinitiate clotting upon “sustained or recurrent” bleeding. The composition of binding agents and clotting agents is suitable to treat more serious external wounds such as those that ordinarily require pressure to stop or reduce the bleeding. Advantageously, the wound sealant composition of a binding agent and a clotting agent provides a physical barrier to bleeding and acts with the natural fibrinogen found at the wound site by the bleeding resulting in more rapid activation of the clotting pathway and more rapid clot formation.

The hydroxylated silica nanoparticle preparation, optionally having one or more clotting agents, is prepared as a sterile preparation for single-delivery application to a wound site. The preparation is capable of being packaged and supplied in four preferential formulations: dry powder, dry adhesive coating, dry aerosol, or liquid (non-aqueous). The formulations are applied topically to a wound site, or may be introduced internally into the wound site in the case of deeper lacerations or during surgical procedures. In various other aspects, the wound sealant formulation includes clotting agents and binding agents, thus providing a thrombolytic cascade accelerant to the wound site. Preferably, the clotting and binding agents are supplied as a premixed formulation. In various embodiments, the binding agents include the thrombolytic activators thrombin and thromboplastin. Preferred embodiments include recombinant forms of these clotting agents, specifically recombinant human thrombin and thromboplastin, and more preferred embodiments include active fragments thereof. Most preferably, the clotting agents are provided in dried or lyophilized form, and are substantially free of fibrinogen or fibrin-analogs.

In still other aspects, thrombolytic cascade accelerants are used as a adjunct to direct wound site treatment with the formulations described herein, for example administered systemically or locally to a patient concomitantly with the hydroxysilica nanoparticle preparations. Recombinant polypeptides are preferable over purified native or animal materials as they are free of viruses, are of acceptable purity, and have been proven safe and effective.

There are additional features of the invention that will be described hereinafter. In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of the description and should not be regarded as limiting.

A primary object of the present invention is to provide a family of wound sealants that exploit high surface area, highly hydrophilic silica nanoparticles (typically from fumed silica) to create lattice structures, stimulate processes, and create additive opportunities, all to improve and accelerate blood clotting process beyond the capabilities of prior art materials and methods.

Another object of the present invention is to provide a one-step delivery wound sealant using silica nanoparticles that agglomerate into chains when hydrated by the aqueous component of bleeding, one result of which is thixotropy of the wound fluids.

Another object is to provide a single-component/single-delivery wound sealant that, when hydrated, creates a fabric of agglomerated chains of silica nanoparticles in situ, to capture red cells and other blood components and to impede their flow from the wound site.

Another object is to provide a one-step wound sealant that immediately seals and stops bleeding even to the capillary level, due to the nanometer dimensions of the agglomerated silica nanoparticles that chain from fibers into a three dimensional lattice.

Another object is to provide a one step wound sealant using nanoparticles of silica that is adaptable to a variety of single-delivery modes and media (dry, liquid, coating on patch/bandage, foam, aerosol), thus avoiding pre-wetting, pre-mixing, or activation time delay.

Another object is to provide a one step wound sealant consisting of a network of silica nanoparticles, with all the advantages and features of the single-component/single-delivery sealant, to which materials and substances can be added to enhance clotting.

Another object is to provide a one step wound sealant containing human recombinant thrombin and thromboplastin to accelerate the thrombolytic cascade in the case of deep wound or internal bleeding.

Another object in the case of external bleeding wherein excess fluid is released is to provide a one step wound sealant consisting of silica nanoparticles dispersed with or coated onto other molecular water absorbents of larger particle size so as to keep the ratio of nanoparticles to fluid at the wound site within a certain ratio favoring lattice formation, viscosity, and degree of thickening. Such absorbents may include inert materials of high water binding capacity such as silicaeous perlite or vermiculite, molecular sieve alumina or alumina silicate microspheres, or alumina gels, ceramic microspheres, porous non-activated or activated carbon as absorbent, or the like.

Another object is to provide a one step wound sealant consisting of a network of silica nanoparticles to which various other clotting factors, calcium cations, astringents, accelerants, fibers, absorbents or adsorbents, antimicrobials, and other components can be admixed to enhance clotting and optimize the material for different types of wounds, patients, environments, and hematological requirements.

Another object is to provide a one step multiple-component wound sealant formulation that is not susceptible to self-activation or interaction between the components while the formulation is in storage. Another object is to provide a one step multiple-component wound sealant that has a useful storage life, and that requires minimal special packaging and/or storage conditions. Another object is to provide a wound sealant using materials with cost-effectiveness superior to that of methods described in the prior art.

Another object is to provide a wound sealant of nanoparticles of silica that uses inexpensive material from an inorganic source, thus reducing costs.

Another object is to provide a wound sealant based fundamentally upon nanoparticles of silica that form a pliable wound dressing when applied, with the ability to respond to movement without being damaged, and to permit treatment of almost any size wound.

Another object is to provide a wound sealant using nanoparticles of silica that provides sustained clotting at the application site due to the ability of the material to reform, permitting the handling of continuous or renewed bleeding.

Another object is to provide a wound sealant using nanoparticles of silica that is effective against serious trauma involving pressure (arterial) bleeding, not only to effectively stop the bleeding, but also to accommodate and utilize the clotting factors naturally present in the body fluid present at a wound site.

Another object is to provide a wound sealant that is an efficient transport vehicle for thrombolytic cascade accelerants and various clotting factors that may be incorporated as required into the tissue sealant formulation to facilitate control of pressure bleeding.

Another object is to provide a wound sealant that is an efficient transport vehicle for thrombolytic cascade accelerants that are animal-derived or recombinant-derived in admixture with the binding agent.

Another object is to use thrombolytic cascade accelerants composed of non-interactive components (not directly reactive with each other in the blood clotting process) such that the components can be formulated without concern for reaction or cross-reaction upon contact and formulation, and for which there is no need to keep components separate for fear of contact and “firing” the system.

Another object is to supply those thrombolytic cascade accelerant components naturally present in the body, as part of the thrombolytic cascade, that are found at relatively low, rate-limiting, serum concentration so as to accelerate rather than limit or scavenge the clotting process, versus those components found at relatively high concentrations and ready abundance in the serum.

Another object is to provide an excess of critical core thrombolytic cascade accelerant precursor components involved in the activation of plasma components already supplied by the body as building blocks for clot formation vs. supplying those essential clot formation dependent factors themselves (i.e. prothrombin or fibrinogen).

Another object is that those thrombolytic cascade accelerant precursor components be involved in “activation” of the cascade at critical rate-limiting steps such as would occur with catalytic enzymatic processes.

Another object is to provide a wound sealant comprised of thrombolytic cascade accelerants supplied at greater-than physiological conditions.

Another object is to provide a wound sealant with thrombolytic cascade accelerant reagents that are stable, yet immediately bioactive in liquid form, including non-aqueous liquid formulations.

To achieve these and other objects, the invention is hereby summarized. In one aspect, the invention includes a wound sealant composition having a binding agent including a plurality of reactive silica nanoparticles having surface hydroxyl groups. The silica particles agglomerate into supramolecular lattice of hydrogen-bonded chains of silicon dioxide when applied to a bleeding wound. In one embodiment, the silica nanoparticles have a surface area of greater than about 400 M²/g. In another embodiment, the silica nanoparticles have a surface area of about 25 M²/g to about 500 M²/g. In another embodiment, the silica nanoparticles have an average diameter of about 0.1 nanometer to about 100 nanometers. In another embodiment, the silica nanoparticles have an average diameter of about 1 nanometer to about 10 nanometers.

In another aspect, the invention provides a dual-component wound sealant composition that includes, at least a binding agent further having a plurality of sterile silica nanoparticles having surface hydroxyl groups, and a clotting agent. The silica particles agglomerate into supramolecular lattice of hydrogen-bonded chains of silicon dioxide when applied to a bleeding wound, and the clotting agents accelerate hemostasis by activating the clotting cascade. In one embodiment, the clotting agent is an extrinsic factor. In one embodiment, the clotting agent is an enzyme. In one embodiment, the clotting agent is thrombin or a thrombolytic fragment thereof. In one embodiment, the clotting agent is recombinant human thrombin or a thrombolytic fragment thereof. In one embodiment, the clotting agent is thromboplastin or a thrombolytic fragment thereof. In one embodiment, the clotting agent is recombinant human thromboplastin or a thrombolytic fragment thereof. In one embodiment, composition has from 1 about microgram to about 1 milligram of clotting agent per about 10 mg of silica nanoparticles. In one embodiment, the composition has from about 10 micrograms to about 500 micrograms of clotting agent per about 10 mg of silica nanoparticles. In one embodiment, the composition has from about 100 micrograms to about 250 micrograms of clotting agent per about 10 mg of silica nanoparticles. In one embodiment, the silica nanoparticles have a surface area of greater than 400 M²/g. In one embodiment, the silica nanoparticles have a surface area of about 25 M²/g to about 500 M²/g. In one embodiment, the silica nanoparticles have an average diameter of about 0.1 nanometer to about 100 nanometers. In one embodiment, the silica nanoparticles have an average diameter of about 1 nanometer to about 10 nanometers.

In another aspect, the invention provides a method of inhibiting bleeding in a mammal comprising, applying to a mammalian subject having a bleeding wound, an effective quantity of the wound sealant composition described, thereby inhibiting the bleeding from the wound and optionally inducing the clotting cascade to initiate hemostasis in the subject.

In another aspect, the invention provides a method of making a wound sealant composition comprising, obtaining a plurality of silica nanoparticles, hydroxylating the silica nanoparticles, and sterilizing the hydroxylated silica nanoparticles, thereby obtaining a wound sealant composition. By admixing the sterilized hydroxylated silica nanoparticles with a second compound such as an excipient, a surfactant, a resin, an antibiotic, an absorbent, an enzyme involved in clotting pathways, an antifungal agent, an antiseptic, polyfunctional short-chain molecules and a mordant, various embodiments of formulations are provided. In one embodiment, the invention includes conjugating to the sterilized hydroxylated silica nanoparticles, a clotting agent. In one embodiment, the clotting agent is an extrinsic factor. In one embodiment, the clotting agent is an enzyme. In one embodiment, the clotting agent is thrombin or a thrombolytic fragment thereof. In one embodiment, the clotting agent is recombinant human thrombin or a thrombolytic fragment thereof. In one embodiment, the clotting agent is thromboplastin or a thrombolytic fragment thereof. In one embodiment, the composition has from about 1 microgram to about 1 milligram of clotting agent per about 10 mg of silica nanoparticles. In one embodiment, the composition includes from about 10 micrograms to about 500 micrograms of clotting agent per about 10 mg of silica nanoparticles. In one embodiment, the invention includes from about 100 micrograms to about 250 micrograms of clotting agent per about 10 mg of silica nanoparticles. In yet another aspect, the invention provides a process for manufacture of a medicament comprising preparing a wound sealant composition, wherein the wound sealant is suitable for treating excessive bleeding in a subject in that it promotes hemostasis and clotting when applied to the wound site of a subject having a wound.

The wound sealant preparations described herein have applications in ameliorating or reducing bleeding from a wound site in a subject, preferably a human, although one of skill in the art will realize that veterinary applications are applicable. The wound sealants thus provides various methods of regulating hemostasis in a subject. The wounds treatable by the various formulations include topical wounds, deeper wounds, and surgical incisions, among others. Accordingly, the various applications of the wound sealants include first aid and triage applications, and medical procedures. Other objects and advantages of the present invention will become obvious to the reader and it is intended that these objects and advantages be within the scope of the present invention.

To the accomplishment of the above and related objects, this invention may be embodied in the form illustrated in the accompanying drawings, attention being called to the fact, however, that the drawings are illustrative only.

### DESCRIPTION OF THE FIGURES

FIG. 1 illustrates the blood coagulation cascade. Both the intrinsic and extrinsic pathways are shown.

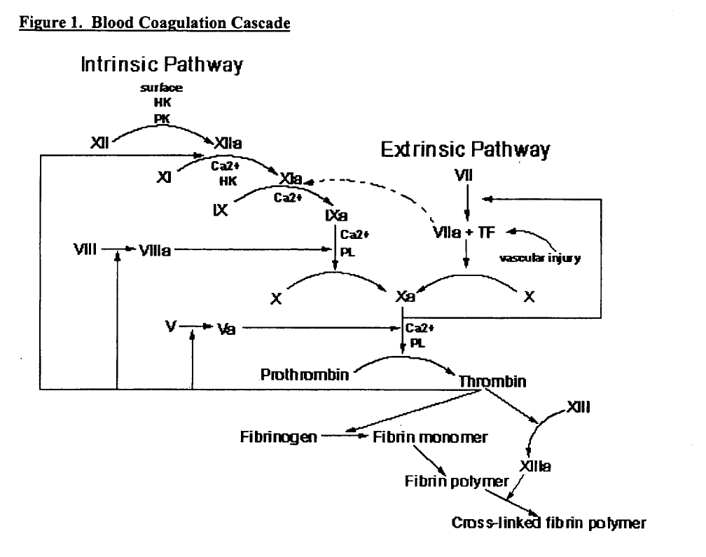

FIG. 2 illustrates the mode of action for a single component wound sealant, comprising a preparation of hydroxylated silica nanoparticles (binding agent).

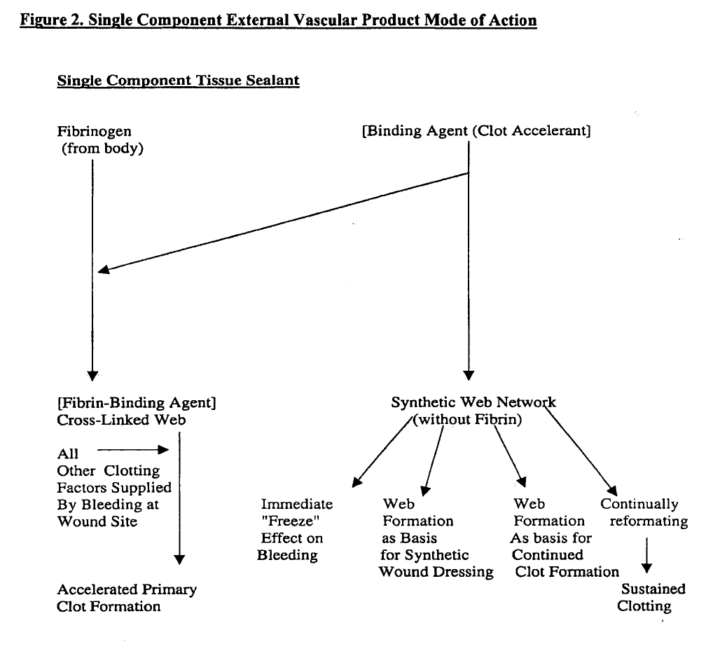

FIG. 3 illustrates the mode of action for a dual component wound sealant, comprising a preparation of hydroxylated silica nanoparticles and (binding agent) and various clotting agents.

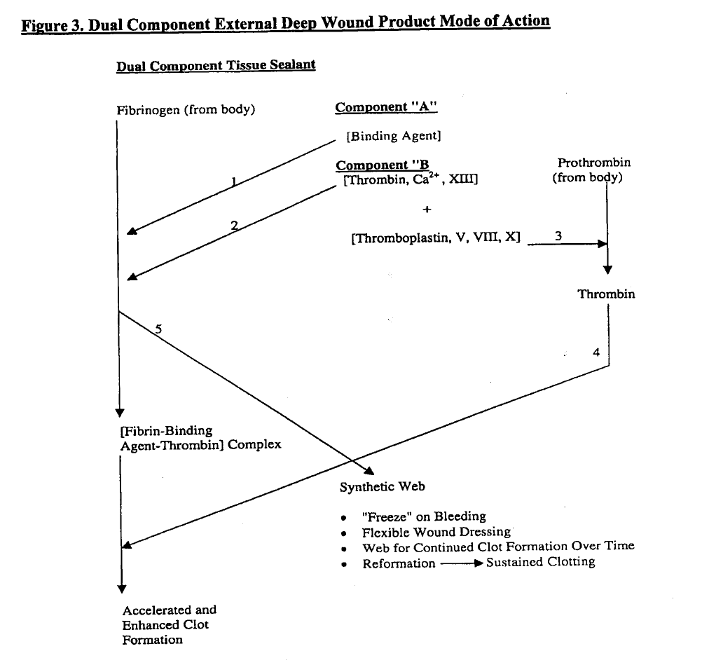

### DETAILED DESCRIPTION

For the external bleeding applications, the binding agent, is comprised of sterile fumed silica nanoparticles in short chains with individual surface areas up to about 500 square meters per gram, and preferably with individual particle sizes as small as a few nanometers in diameter. Such silica particles are produced by several processes, of which the most common is the “fumed silica” production technique by Cabot, the “silica fume” production technique by Elkin, and similar products from other companies. Medical grade fumed silica for human use referred to herein is relatively rare (e.g., Cabot sells CAB-O-SIL grades M5 or M5P suitable for human applications). For other applications, whether human or veterinary, where medical grade quality is not as critical e.g., life threatening trauma or battlefield conditions, the use of Cabot grades L-90, LM-130, LM-150, PTG, M-7D, MS-55, H-5, HS-5, or EH-5 may be used. All grades fall within the range of 90-380 M²/g average surface area, less than 0.02% 325 mesh residue (44 microns), a size less than 100 nanometers, and have appropriate reactive surface chemistry.

During the fuming process used to prepare the nanoparticles numerous surface hydroxyl groups are produced on the surface of the particle. This renders the particles highly hydrophilic, another feature that contrasts it with other conventional silica microparticles. Two types of hydroxyl groups are generally produced on the surface of nanoparticles when prepared by the fumed technique. Fumed silica is produced by hydrolysis of silicon tetrachloride in a hydrogen oxygen flame at 1800 degrees C. which results in silicon dioxide molecules which upon condensation produce nanoparticles with surface hydroxyl groups. Observed by IR are two two types of hydroxyls: isolated hydroxyl groups with an absorption maxima at 3750 cm-1 which are highly hyrdrophilic; and hydrogen-bonded hydroxyl groups (3700 to 3500 cm-1) that are also highly hydrophilic. The latter result from the presence of hydroxyl groups attached to neighboring surface silicon atoms. The surface density of hydroxyl groups could be theoretically as high as ˜8 hydroxyl groups per square nanometer if all silicon atoms had one hydroxyl, but the average tends to be 4 hydroxyls per square nanometer by chemical and thermogravimetric analysis.

It may be possible to improve upon the surface hydroxylation of nanospheres as noted in Langmuir 20:260-262, 2004, Gole, J L et al. It may be possible to adjust the surface oxidation states in Si/SiO2 nanoparticles through the ratio of metalloidions/metalloid ions in the starting mixture. As example, variation of the ratio of Si4/SiO in the starting mixture may yield nanoparticles more reactive to the phenolic hydroxylation reaction resulting in fine tunable average surface oxidation states.

To facilitate appreciation of the preferred embodiment of this invention, certain of its characteristics and functions should be explained. When hydrated, the binding agent instantly agglomerates into a supramolecular network, or fabric, of cross-linked chains of silicon dioxide, in a lattice form that provides a three dimensional framework for clot formation with dimensions below one micron to permit effectiveness at every level of bleeding down to the capillary. The water present in the blood and serum of the wound site participates in the creation of the lattice thus serving two purposes: three dimensional lattice formation resulting in small pore sizes for entrapment of blood cells and clotting factors and flow control; and water absorption (by hydrogen bonding as part of the lattice structure itself) resulting in thickening. This lattice will also cause the fluid to become a thixotropic gel in the absence of sheer forces, therefore serving as a flexible wound dressing that continually reforms itself in response to sheer forces and the availability of additional body fluid at the wound site. Though the binding agent is by itself a useful wound sealant, it is also a convenient non-interactive carrier of other components to enhance the clotting and wound-sealing processes.

The binding agent is therefore a stand-alone, single-component/single-delivery sealant comprised of silica particles, prepared as a sterile material. These particles are a few nanometers in diameter, and have surface groups of hydroxyls and siloxanes capable of hydrogen bonding at the site of application. Hydroxyl groups are known to irritate platelet membranes in wounds with the subsequent release of clotting factors. Free hydroxyl groups in a wound produce a sting reaction owing to the caustic alkali. In this formulation, however, the hydroxyl groups are found on the silica surface at high density and serve to attract and entrap platelets but do not produce the sting reaction at the wound site as is noted with certain oxyacid preparations that require addition of a cation exchange material to offset the sting reaction (20020141964). This is viewed as a beneficial feature. Upon aqueous hydration by body fluids, the binding agent immediately creates a web formed through hydrogen bonding that both provides a matrix for clotting and makes the aqueous component of the blood thixotropic, to reduce flow, in addition to the attraction of platelets with release of clotting factors.

Silica can be used as long or short chains of agglomerated nanoparticles ranging in surface area from 25 square meters per gram to t five-hundred square meters per gram or greater but more preferably ˜200 square meters per gram. The degree of network formation is dependent upon several factors that can be controlled either through the formulation and compounding or in the method of application at time of use. Obviously the concentration and grade of nanoparticle influence three dimensional network formation. The grades and concentrations described in this patent-have been found to work. The pH in the wound site is also important. A pH of greater than 2.3 up to 8 is suitable, preferably between pH 5 to 7. The isolectric point for nanosilica is approximately 2.3 where it is electrically neutral. Most blood samples have pH's between 4 and 9. The degree of dispersion in a blood sample is also important. The high hydrophilicity of reactive silica nanoparticles for water in a wound site routinely assures the ‘draw-in’ of aqueous fluid into the admixture once applied as a powder to the skin. This assures adequate and rapid dispersion. The use of non-aqueous based liquid formulations is also effective as aqueous fluid from the wound is drawn into the admixture as solvent evaporates from the skin surface above the wound site assuring adequate dispersion.

Binding agent can be applied as a powder or as a coating, or blended with a non-aqueous low hydrogen bonding liquid or solvent at any concentration from under 0.1% to over 99.9%. It may also be blended with non-hydrogen-bonding materials such as aliphatic hydrocarbons (mineral oil) wherein other additives in the wound sealant formula may be coadsorbed to the nanoparticles for ready delivery to the wound site. Upon contact with highly polar water within the fluid of the wound, the coadsorbed materials are delivered to the fluid phase in exchange for nanoparticle hydrogen bonding to water molecules. This would help facilitate dispersion of wound sealant additives, such as antibiotics, analgesic's or other medications. The binding agent can be delivered as a dry powder, or in a non-aqueous liquid carrier. It can be added to bandages as a non-aqueous gel, or as a powder. The binding agent can be admixed with a dry inert carrier such as talc, or a similar material, or coated onto or incorporated into any conventional wound dressing material.

To the silica nanoparticles, various soluble or insoluble, synthetic or naturally occurring short chain monomers or polymers may be added to the mixture in dry form. Although not required for lattice formation, these materials may be entrapped within the lattice itself further strengthening the web network in situ acting as a mordant (cement) between the cross-linked silica framework. Materials hereby incorporated by reference include but are not limited to: cross-linked anionic or cationic polyamine or polyacrylamide flocculent material (PAMS); lignosulfanates; hyaluronan; synthetic polyketides; polyhydroxyalkanoates, cutin or suberin digests of plant material (naturally occurring polyesters); poly(g-D-glutamate); polymerized human serum albumin (recombinant); bioplastic polymers like pullanan and the fungal polymer scleroglucan; and naturally occurring non-edible polysaccharides like dextran, and the like. Protein polymers including collagen and fibrinogen are also useful.

Dry, flocculent, neutral, anionic or cationic, cross-linked polyamine, polyDADMAC, or polyacrylamide (Cytec, Inc., SUPERFLOC) could be used for fluid absorption or to aid as a mordant and are available in a variety of MW's of varying viscosity. Lignosulfates are naturally occurring GRAS (Generally Regarded As Safe) materials extracted from wood pulp by various processes and are used in animal feeds and as indirect food additives. They occur in polymeric form following digestion and are hydrophilic and are used as adhesives, binders and sequestrants. Hyaluron is a GRAS linear polysaccharide used in cosmetics.

Silica nanoparticles form a three dimensional lattice network within the fluid of a wound sample over a wide range of particle mass to fluid volume ratios. In the event of excess bleeding or excess fluid at a deep wound site it may be advantageous to mix silica nanoparticle dry powder with other materials that can function as water absorbents as an activity secondary to lattice formation to facilitate the take up of fluid within the wound to aid the thickening of the sample to facilitate clotting by enhancing the proximity of components or could serve the opposite purpose of intentionally keeping the wound hydrated (wet) to control the moisture loss rate. Such properties may be especially useful in burn victims to control fluid loss rate. Owing to its high surface area it may be advantageous to mix silica nanoparticle powder with various conventional large particulate water adsorbent materials at varying ratios to facilitate wound fluid absorption. Such absorbents include ultra fine ground perlite (1600° C. heated silicaeous volcanic rock; 200-600% water absorption, % weight); ground heat expanded vermiculite (220-325% water absorption, % weight; 4-Superfine grade; 90-160 Kg/M³ density); cross-linked agarose gels such as Sephadex® and or Sepharose®; synthetic molecular sieve powders such as Purmol®; molecular sieve alumina, or alumina gels, or alumina silicate microspheres used in deodorants (Lawrence Laboratories; UOP); ceramic microspheres, zeolites, and/or inactive or activated carbon or charcoal. All these materials at least double their weight with water and are compatible with the binding agents.

Aluminas are a family of synthetic aluminum oxide beaded powders that have specific rheological and absorbent properties. Typical synthetic adsorbents such as UOP International's Versal Aluminum oxides (A 1203), so called gel aluminas are examples, in addition to UOP's molecular sieves (MOLSIV powder).

As a single-component/single-delivery system, the binding agent produces an immediate “freeze” effect upon blood flow due to its thixotropic effect upon the aqueous constituents of the blood and the creation of a web fabric that captures blood cells, and the resulting clot consists of a synthetic wound dressing that supports continued clot formation. The fluid of the wound contains fibrinogen from the body, which meets the cross-linked web along with blood cells and plasma, containing all other clotting factors ordinarily provided by bleeding, and collectively accelerates primary clot formation. The clot will reform as necessary at every level even with dimensions below one micron, maintaining coverage and sealing of wounds and bleeding channels even from capillaries.

In the embodiments containing clotting agents, the same lattice formation processes occur, with the same advantages. However, the optional addition of clotting agents, such as human recombinant clotting components (some combination of thrombin, thromboplastin, or various other factors, cations, etc.) biochemically accelerates the thrombolytic cascade to produce a further improvement in the speed of clot formation and wound sealing. The clotting agents are admixed with the binding agents, or adsorbed to the surface of the nanoparticles through hydrogen bonding. Subsequent reaction with more polar water from the wound site would result in simple release of the adsorbed factors to allow ready solubility and subsequent reactivity.

A two-part wound sealant preparation consists of the same silica nanoparticle preparation, to which a second component representing any one, or a combination of several, clotting agents including thrombolytic cascade accelerant(s), has/have been admixed. The clotting agents are native derived or preferably recombinant thrombin and thromboplastin, prepared by any of several methods. The clotting agents can be dried or lyophilized in advance to form a grindable or dispersable powder; dried or lyophilized after addition to a non-aqueous formulation containing a defined percentage of a non-hydrogen binding liquid such as glycerol so as to form a grindable powder; dried by evaporation after addition to a non-aqueous, non-hydrogen binding solvent such as certain alcohols. The use of non-hydrogen binding materials is required to avoid interactions between the silica nanoparticles in storage.

The source of the thrombolytic materials selected will be determined by the host it is used on. For example, veterinary formulations may use animal derived materials. It is preferred that the thrombolytic cascade accelerant be free of fibrinogen or fibrin-analog, and consists of thromboplastin and thrombin to activate cleavage of natural fibrinogen found at the wound site, thus producing fibrin and leading to the desired thrombolytic cascade. The thromboplastin is selected from a wide range of sources including simplastin, thromboplastin reagent, brain thromboplastin, British comparative thromboplastin, Thromborel S, calcium thromboplastin, porcine brain thromboplastin, ox brain thromboplastin, Innovin R, Recombiplastin, and others of similar characteristics. The preferred material is recombinant human thromboplastin. The thrombin (r-thrombin) is typically from activated recombinant human thromboplastin from human CHO cells using Hirudin and Hirudin-based peptide sepharose chromatography or produced by recombinant techniques known in the art.

Recombinant human thrombin and thromboplastin are available and are the reagents of choice for human use. The formulation is designed to be stable in both liquid and dry form, yet retaining and maintaining its specific reactivity and bioreactivity at peak levels. It is also formulated to maintain full functionality in the presence of the binding agent without interaction between the two components, or impediment of the hydrogen-bonding web formation by the binding agent. The wound sealant having clotting agents is formulated with a non-hydrogen-bonding liquid, e.g. mineral oil, wherein and preferably the clotting agents are adsorbed to the hydroxysilica nanoparticles. Alternatively the clotting agents can be introduced as an admixture of low hydrogen-bonding polyfunctional short chain molecules, e.g. polyethylene glycol 3350, polyoxyethelene-6-sorbitol, or non-ionic surfactants such as polysorbate 60, in non-aqueous liquid form combined with thrombin and thromboplastin.

At the wound site, any weakly hydrogen-bonded thrombin or thromboplastin molecule coadsorbed to polyfunctional short-chain molecules or non-ionic surfactants will immediately release materials to hydrolysis upon primary hydration of the active silica nanoparticle carrier with the highly polar water available in the ambient body fluid. This will result in the preferential binding of the hydroxyl groups on silicon dioxide (binding agent) to the more highly polar water molecules as the basis for web formation. This allows the clotting agents to be released into the fluid for accelerated clot formation, thus creating a one step, single-delivery, liquid admixture tissue sealant.

Fragments of clotting agents can be used as an alternative to using the whole polypeptide. Thrombin is not just an enzyme with moderately restricted proteolytic capabilities, yet with extraordinarily high specificities for certain bonds (such as the A alpha-cleavage site in fibrinogen), but also is a protein with hormonelike activities involving cell receptor interactions. Such activities do not require the catalytically active enzyme, but are blocked by hirudin (also antithrombin III). These appear to involve an unique insertion and subsequent peptide segment at an exon junction. On the other hand, the enzymatic functions of thrombin depend on the catalytic site, per se, and derive specificity from the adjacent apolar-binding site within the fibrinopeptide side and the independent anionic-binding site within the fibrin side of the active groove. See, Fenton, J W et al., Thrombin active-site regions. Semin Thromb Hemost. 1986 Jul;12(3):200-8, for a discussion of the specific thrombin peptide regions that are involved in the clotting pathway and are suitable thrombin peptide fragments for conjugation to binding agents as described herein. See, McCallum et al., J Biol Chem. 1996 Nov 8;271(45):28168-75, for a discussion of specific thromboplastin peptide regions that are involved in the clotting pathway and are suitable thromboplastin peptide fragments for conjugation to hydroysilica nanoparticles as described herein.

Additional clotting factors involved in clot formulation may be supplied as part the tissue sealant or simply provided by the body at the site, though they are not critical to effectiveness. They may be purified native (human or animal), or recombinant materials. For the thromboplastin mediated reaction, Factors V, VII, and X may be additionally supplied. In addition to this, or alternatively by itself, Factor XIII may be additionally supplied resulting in a thrombin-mediated clotting reaction. Likewise, various methods or improvements known in the art may be integrated or included in the wound sealant preparations disclosed herein. As an example, the formulation described above may be modified to provide a liquid stable thrombin through use of a polyol or other stabilizer (EPS 0277 096B1), addition of plasmin inhibitors (U.S. Pat. No. 5,645,859), or inclusion of other blood clot techniques known in the art.

The clotting agents stimulate typical thrombin-like proteases supporting fibrinogen cleavage to fibrin. These permit the wound sealant's use in applications with heavy bleeding, trauma use, and applications of recurrent bleeding, even in cases of hemophilia, and even where the subject may be taking doses of blood thinners and anti-clotting agents. The two basic building blocks of the clot, namely prothrombin and fibrinogen, are supplied by the body at the wound site in relatively high levels. The clotting agents in the wound sealant preparation accelerate and catalyze the clotting process and use these naturally available clot proteins, the clotting effect working in parallel and tandem with the activated binding agent, which provides a matrix or lattice that traps blood cells and plasma for enhanced hemostasis.

The appropriate concentrations of thrombin and thromboplastin will obviously depend on whether the formulation is prepared for severe or more moderate bleeding. Generally enzyme concentrations per dose of a liquid dual-component wound sealant formulation will range from 0.01 nanomolar to 10 micromolar of clotting agents, preferably 0.1 to 1000 nanomolar concentrations, more preferably 1 to 100 nanomolar concentration, and most preferably about 10 to 50 nanomolar concentrations of clotting agents. In a dry formulation, enzyme weights per dose of a powder/lyophilized dual-component wound sealant formulation will range from about 1 nanogram to 100 mg of clotting agents, preferably 10 nanograms to 10 miligrams, more preferably 100 nanograms to 1 miligram clotting agents, and most preferably about 1 microgram to 100 micrograms of clotting agents. Modifications to the specific concentrations of each clotting agent will be apparent to those of skill in the art, given published activities of the various clotting cascade enzymes at numerous concentrations. See, Lo K, Diamond S L, Blood coagulation kinetics: high throughput method for real-time reaction monitoring., Thromb Haemost. 2004 Oct;92(4):874-82. Clotting agents should not saturate the silica nanoparticle surfaces; the hydrogen bonded lattice structure is desirable.

An appropriate dose of a wound sealant will depend largely on the particular injury type, and can be assessed by a medical professional. Additionally, a subject with a clotting deficiency or disorder, or one taking blood thinner medications may require additional quantities of the appropriate formulation. By way of non-limiting example, a 2-cm laceration characterized by small-vessel bleeding may be treated using 1-100 mg or more of a powder formulation. A small puncture wound e.g., from a needle or lancet stick may be treated using 1 -10 or more mg of a powder formulation or 1 drop of a liquid formulation. Deep wounds may be packed with gram quantities of a sterile dry powder formulation, or with varying weights of single and dual-component dry formulations. Single component wound sealant preparations are essentially silica, and are generally inert in the body.

### EXAMPLE ONE

### Powder Bandage Formulation

The dry powder formulation for external use as a consumer applied powder for simple vascular bleeding employs two main ingredients. In a suitable mixing container, medical grade aluminum sulfate, Al₂(SO₄)₃ powder (Sigma) is admixed with CAB-O-SIL grade M5 powder (Cabot) to a final concentration of 1.0 percent on a wt/wt basis. It is preferable that mass be determined gravimetrically. It is preferable to compound the materials at <40% RH at ambient room temperature using stainless steel containers on a grounded steel table with antistat mats on the floor to minimize static charge as a result of mixing. Although charge will not adversely effect the product, lack of control may make GMP compounding messy as the silica material is extremely light. Mixing must also be slow wherein aluminum sulfate should be added to silica first and not in the reverse order. Following adequate mixing the material is dispensed into a suitable container for market and sealed. If required, the material can be sterilized by gamma irradiation or other medically acceptable sterilization techniques such as ethylene gas and autoclaving.

To prepare a powder for external use for deep bleeding containing both clot accelerant and thrombin and thromboplastin, to the above mixture add dry (dried or lypholized, both as powder) recombinant thrombin and thromboplastin powder gravimetrically to a final concentration of up to 2% each, preferably 0.5% wt/wt. The aluminum sulfate can be removed from this formulation if desired for internal use. After filling, the material may be sterilized by gamma irradiation. Preferred dry dual-component wound sealant formulations include but are not limited to, having from 1 microgram to 1 milligram of clotting agent per 10 mg of silica nanoparticles, from 10 micrograms to 500 micrograms of clotting agent per 10 mg of silica nanoparticles, and most preferably having from 100 micrograms to 250 micrograms of clotting agent per 10 mg of silica nanoparticles.

### EXAMPLE TWO

### Liquid Bandage Formulation

Two phases are provided, one liquid, one solid, which are admixed into a single formulation as a liquid bandage. The preparation is stored as a liquid. Solid component is comprised of reactive fumed silica nanoparticle powder, grade M-5P (Cabot), 0.1-99.9% wt/vol, or equivalent, preferably 20% wt/vol. Solid phase is admixed into liquid phase. Liquid phase is comprised of a non-aqueous evaporative solvent based solution of pyroxylin or other polymeric materials. Pyroxylin is a generic name for cellulose nitrate resin compounds that form a film when dissolved in a mixture of solvents like ether and alcohol. After suitable mixing by stirring the admixture is dispensed into a suitable container (plus lid) for consumer use.

After application at the wound site, evaporation (quick drying) takes place leaving a thin film on the surface. To facilitate evaporation of product after application solvents include; acetone, ether, amyl acetate (Banana solution), alcohol (methanol or ethyl alcohol), etc and various combinations thereof. A variety of polymeric materials can be used in the liquid phase.

The following are nonlimiting examples of various cellulosic resins that can be used: cellulose nitrate (nitrocellulose), cellulose acetate butyrate, cellulose acetate proprionate, cellulose acetate, cellulose proprionate, ethyl cellulose, carboxy methyl celluose.

The following are nonlimiting examples of various non-cellulosic resins that can be used: polymeric dextran, cross linked polyamine or polyacrylamide flocculants

(PAMS), polyhydroxyalkanoates, cutin or suberin digests of plant material (naturally occurring polyesters), synthetic polyketides.

Mechanism of Action

The non aqueous liquid solvents that are used are mildly polar and evaporate quickly when applied to the wound site. The hydroxyl groups on the silica dioxide nanoparticles are attracted to the more highly polar water molecules and to themselves to form a lattice framework upon drying. The material that is applied both penetrates and interacts with and covers the wound site like a clear plastic bandage. This provides not only a lattice framework for clot formation but a plastic bandage covering at the wound site.

In dual-component liquid formulations, clotting agents are admixed with silica nanoparticles. They can be crosslinked to the binding agent, but preferably they are allowed to hydrogen-bond to the silica nanoparticles. In other embodiments, they are optionally adsorded to excipients and are mixed with the biding agent into a liquid base. For example, a thrombin or thromboplastin molecule is coadsorbed to polyfunctional short-chain molecules or non-ionic surfactants, and the preparation when applied to a wound site will immediately release clotting agents to hydrolysis upon primary hydration of the active silica nanoparticle or carrier with the highly polar water available in the ambient body fluid. This will result in the preferential binding of the hydroxyl groups on silicon dioxide (binding agent) to the more highly polar water molecules as the basis for web formation between the silica nanoparticles. Preferred liquid dual-component wound sealant formulations include but are not limited to, having from 1 microgram to 1 milligram of clotting agent per 10 mg dry weight of silica nanoparticles, from 10 micrograms to 500 micrograms of clotting agent per 10 mg dry weight of silica nanoparticles, and most preferably having from 100 micrograms to 250 micrograms of clotting agent per 10 mg dry weight of silica nanoparticles.

The elastopolymer pyroxylin resin will not wash off for several days. Additives can be added to the admixture, for example but not limited to antiseptics such as 8-hydroxyquinoline alcohol and iodine, etc., antibiotics such as polysporin, neosporin, penicillin, methicillin, cephalosporin, erythromycin, vancomycin, gentamycin, ciprofloxicin and other broad spectrum antibacterials, antifungal agents such as terbinafine and amphotericin, and other absorbents and mordants as described above. These additives also work well in the dry formulations. If required, the liquid formulation can be sterilized by gamma irradiation or other medically acceptable liquid sterilization techniques.

The foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, all falling within the scope of the invention. This includes various embodiments that include the various compositions and methods described in the various art references cited and thus incorporated herein by reference in their entirety.

### EXAMPLE 3

### Clotting Study

To determine the efficacy of the formulation described in Example 1, the following adult volunteer study was conduct. All participants were apparently healthy normal adults with no history of bleeding disorders and not on blood thinning agents. Depending upon the dexterity of the individual either the right or the left forearm or calve was used. A small needleprick was made using a lancet in two duplicate spots and gently expressed to induce uniform minor bleeding at the wound site as would occur upon puncture or alternatively a raspy file was dragged across the skin to abrade it to induce minor bleeding as would occur upon abrasion.

Immediately after puncture or abrasion, in either case, dry powder (Example 1, without thrombolytic factors) was sprinkled generously onto one of the two cut sites. Care was taken to generate comparably sized cuts. Excess powder was shaken off after 45 seconds and relative clotting time, relative scab tightness and uniformity after 24 hours, and relative duration of the scab till it fell off were recorded.

The following results were observed:

| | Time to Initial | | Scab Tightness | | Scab | |
|---|---|---|---|---|---|---|
| | Clot (minutes/ | | (24 hr) | | Duration | |
| | seconds) | | (Scale 1-6*) | | (Days) | |
| | Un- | | Un- | | Un- | |
| Volunteer | treated | Treated | treated | Treated | treated | Treated |
| A, abrade | 2 m 20 s | 45 s | 3 | 5 | 4 | 3 |
| B, puncture | 3 m 30 s | 60 s | 3 | 6 | 5 | 4 |
| C, puncture | 2 m 45 s | 50 s | 3 | 6 | 5 | 4 |
| D, puncture | 3 m 10 s | 55 s | 4 | 6 | 4 | 3 |
| E, abrade | 3 m | 70 s | 2 | 5 | 3 | 5 |

Scale:
1, Loose, intermittent bleeding, incompletely formed;
2, Soft, easily disruptable;
3, Moderate to firm clot, blood difficult to express;
4, Good clot, uniform, not expressable,
5, Tight, hard clot, strong adherence to skin;
6, Very tight, condensed clot, shrunken into the skin, painful to express or remove.

We claim:

1. A wound sealant composition comprising, a binding agent further comprising a plurality of reactive silica nanoparticles having surface hydroxyl groups.

2. The composition of claim 1, wherein the silica nanoparticles have a surface area of greater than 400 M²/g.

3. The composition of claim 1, wherein the silica nanoparticles have a surface area of 25 M²/g to 500 M²/g.

4. The composition of claim 1, wherein the silica nanoparticles have an average diameter of 0.1 nanometer to 100 nanometers

5. The composition of claim 1, wherein the silica nanoparticles have an average diameter of 1 nanometer to 10 nanometers.

6. The composition of claim 1, wherein the silica particles agglomerate into supramolecular lattice of hydrogen-bonded chains of silicon dioxide when applied to a bleeding wound.

7. A wound sealant composition comprising, a binding agent further comprising a plurality of sterile silica nanoparticles having surface hydroxyl groups, and a clotting agent.

8. The composition of claim 7, wherein the clotting agent is an extrinsic factor.

9. The composition of claim 7, wherein the clotting agent is an enzyme.

10. The composition of claim 7, wherein the clotting agent is thrombin or a thrombolytic fragment thereof.

11. The composition of claim 10, wherein the clotting agent is recombinant human thrombin or a thrombolytic fragment thereof.

12. The composition of claim 7, wherein the clotting agent is thromboplastin or a thrombolytic fragment thereof.

13. The composition of claim 12, wherein the clotting agent is recombinant human thromboplastin or a thrombolytic fragment thereof.

14. The composition of claim 7, having from 1 microgram to 1 milligram of clotting agent per 10 mg of silica nanoparticles.

15. The composition of claim 7, having from 10 micrograms to 500 micrograms of clotting agent per 10 mg of silica nanoparticles.

16. The composition of claim 7, having from 100 micrograms to 250 micrograms of clotting agent per 10 mg of silica nanoparticles.

17. The composition of claim 7, wherein the silica nanoparticles have a surface area of greater than 400 M²/g.

18. The composition of claim 7, wherein the silica nanoparticles have a surface area of 25 M²/g to 500 M²/g.

19. The composition of claim 7, wherein the silica nanoparticles have an average diameter of 0.1 nanometer to 100 nanometers

20. The composition of claim 7, wherein the silica nanoparticles have an average diameter of 1 nanometer to 10 nanometers.

21. The composition of claim 7, wherein the silica particles agglomerate into supramolecular lattice of hydrogen-bonded chains of silicon dioxide when applied to a bleeding wound.

22. A method of inhibiting bleeding in a mammal comprising, applying to a mammal having a bleeding wound, the composition of claim 1, thereby inhibiting the bleeding from the wound.

23. A method of making a wound sealant composition comprising, obtaining a plurality of silica nanoparticles, hydroxylating the silica nanoparticles, and sterilizing the hydroxylated silica nanoparticles, thereby obtaining a wound sealant composition.

24. The method of claim 23 further comprising, admixing the sterilized hydroxylated silica nanoparticles with a second compound selected from the group consisting of: an excipient, a surfactant, a resin, an antibiotic, an absorbent, an enzyme involved in clotting pathways, an antifungal agent, an antiseptic, polyfunctional short-chain molecules and a mordant.

25. The method of claim 23 further comprising conjugating to the sterilized hydroxylated silica nanoparticles, a clotting agent.

26. The method of claim 25, wherein the clotting agent is an extrinsic factor.

27. The method of claim 25, wherein the clotting agent is an enzyme.

28. The method of claim 25, wherein the clotting agent is thrombin or a thrombolytic fragment thereof.

29. The method of claim 28, wherein the clotting agent is recombinant human thrombin or a thrombolytic fragment thereof.

30. The method of claim 25, wherein the clotting agent is thromboplastin or a thrombolytic fragment thereof.

31. The composition of claim 30, having from 1 microgram to 1 milligram of clotting agent per 10 mg of silica nanoparticles.

32. The composition of claim 30, having from 10 micrograms to 500 micrograms of clotting agent per 10 mg of silica nanoparticles.

33. The composition of claim 30, having from 100 micrograms to 250 micrograms of clotting agent per 10 mg of silica nanoparticles.

34. A method of inhibiting bleeding in a mammal comprising, applying to a mammal having a bleeding wound, the composition of claim 7, thereby inhibiting the bleeding from the wound.
